(19) United States
(12) Patent Application Publication
(10) Pub. No.: US 2016/0192990 A1
(43) Pub. Date: Jul. 7, 2016

(54) Telescopic light source electrosurgical pencil

(71) Applicant: XINHAIHEXING TECHNOLOGY (DALIAN) CO., LTD, Dalian (CN)

(72) Inventor: Yuping WANG, Dalian (CN)

(73) Assignee: XINHAIHEXING TECHNOLOGY (DALIAN) CO., LTD, Dalian (CN)

(21) Appl. No.: 14/983,415

(22) Filed: Dec. 29, 2015

(30) Foreign Application Priority Data
Dec. 29, 2014 (CN) 201410836051.8
Dec. 29, 2014 (CN) 201420853243.5

(51) Int. Cl. A61B 18/14; A61B 18/18; A61B 19/00

(52) U.S. Cl. CPC A61B 19/5202; A61B 18/148; A61B 2018/00589

(57) ABSTRACT

A telescopic light source electrosurgical pencil comprises a handle with a control circuit internally configured. One end of the handle is connected to a cutter head. Two sides of a joint by which the handle is connected to the cutter head are both provided with an LED lamp I and an LED lamp II parallelly arranged on the handle. The handle is provided with an electric cutting switch and an electric coagulation switch, the other end of the handle being provided with an LED lamp switch for controlling the LED lamps. The handle is further connected to a cable, the cable being connected to a plug connected to an electric cutter machine and a power adapter connector. This telescopic light source electrosurgical pencil employs a structure of parallelly arranged LED lamps for illumination, which effectively improves the luminance of the surgical view and thus brings great convenience to surgeries.

### CROSS REFERENCE TO RELATED APPLICATIONS

The present application claims priority to Chinese Patent Application CN 201410836051.8 filed on Dec. 29, 2014 and to Chinese Patent Application CN 201420853243.5 filed on Dec. 29, 2014.

### TECHNICAL FIELD

The present invention relates to the technical field of medical instruments, and in particular, relates to a telescopic light source electrosurgical pencil.

### BACKGROUND

An electrosurgical pencil is widely applied to surgical operations, and is an electronic surgical instrument which is capable of replacing a mechanical surgical knife to perform tissue cutting. A light source electrosurgical pencil in the prior art is only provided with one LED light source in the design. Therefore, the integral structure is not beautiful, the luminance is small, and the illumination is not uniform. In addition, the light source electrosurgical pencil does not have the function of charging in real time, and the battery is needed for charging the pencil. Because shadowless lamps are used in the operating rooms during the surgical operations, too dark view affects the surgical operations when complicated surgical operations are performed or the surgical operations having a small incision.

### SUMMARY

In view of the technical problem in the prior art, the present invention discloses a telescopic light source electrosurgical pencil, comprising an electrosurgical pencil handle in which a control circuit is internally configured, one end of the electrosurgical pencil handle being connected to a cutter head, two sides of a joint by which the electrosurgical pencil handle is connected to the cutter head being both provided with an LED lamp I and an LED lamp II, the LED lamp I and the LED lamp II being parallelly arranged on the electrosurgical pencil handle, the electrosurgical pencil handle being provided with an electric cutting switch and an electric coagulation switch, the other end of the electrosurgical pencil handle being provided with an LED lamp switch for controlling working of the LED lamps, the electrosurgical pencil handle being further connected to a cable, the cable being connected to a plug connected to an electric cutter machine and a power adapter connector.

The electrosurgical pencil handle is internally provided with a power line which collaboratively works with the LED lamp I and the LED lamp II, the power line being connected to the power adapter connector via the cable.

The power line is capable of integrating a DC power supply line and high frequency electric coagulation and electric cutting loops by using a five-core electric wire structure, and the power line is lead out as a plug having a three-core electric wire structure and a power adapter connector having a two-core electric wire structure.

The electrosurgical pencil handle is internally configured with a PCB board, and the electric cutting switch and the electric coagulation switch are capable of controlling working of the electric coagulation and electric cutting by controlling connection or disconnection of wiring of the PCB board.

An isolation plate for isolating the electric coagulation and electric cutting lines from the LED lamp DC loop is arranged at the electric cutting switch and the electric coagulation switch on the electrosurgical pencil handle.

The cutter head is capable of telescoping within a range of 46 mm to 130 mm, and the cutter head is capable of rotating within 360 degrees.

The cutter head is capable of rotating using a longitudinal axis of the electrosurgical pencil handle as a rotation axis.

The cutter head (1) is capable of cutting in the form of being perpendicular to a surface of a cut body, and forming at least one cut on the surface.

The cutter head is capable of rotating at the at least one cut, and maintaining perpendicular to the surface.

A front end of the cutter head is provided with a non-stick coating, the cutter head is provided with an insulating sleeve, and a heat insulating Teflon sleeve is arranged between the insulating sleeve and the cutter head.

With the above technical solutions, the telescopic light source electrosurgical pencil according to the present invention employs a structure of parallelly arranged LED lamps for illumination, which effectively improves the luminance of the surgical vision and thus brings great convenience to surgeries. In addition, no batter needs to be internally provided in the electrosurgical pencil, while the electrosurgical pencil is capable of providing continuous illumination with stable luminance.

### BRIEF DESCRIPTION OF THE DRAWINGS

To describe technical solutions according to the embodiments of the present application or in the prior art, drawings that are to be referred for description of the embodiments or the prior art are briefly described hereinafter. Apparently, the drawings described hereinafter merely illustrate some embodiments of the present application. Persons of ordinary skill in the art may also derive other drawings based on the drawings described herein without any creative effort.

FIG. 1 is a schematic structural view of a light source electrosurgical pencil according to the present invention;

FIG. 2 is a schematic structural view of a light source electrosurgical pencil according to the present invention;

FIG. 3 is a schematic view of a light coverage region of parallelly arranged LED lamps according to the present invention; and

FIG. 4 is a schematic inner structural view of a light source electrosurgical pencil according to the present invention.

### DETAILED DESCRIPTION

To make the technical solution and advantages of the present invention clearer, the technical solution according to embodiments of the present invention is further described in detail with reference to the accompanying drawings and the specific embodiments.

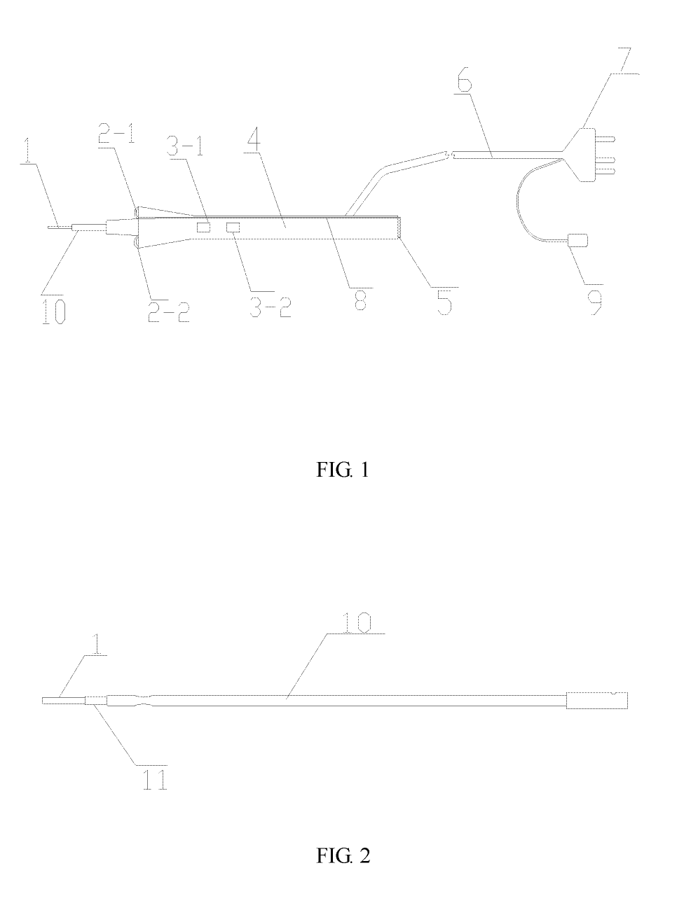

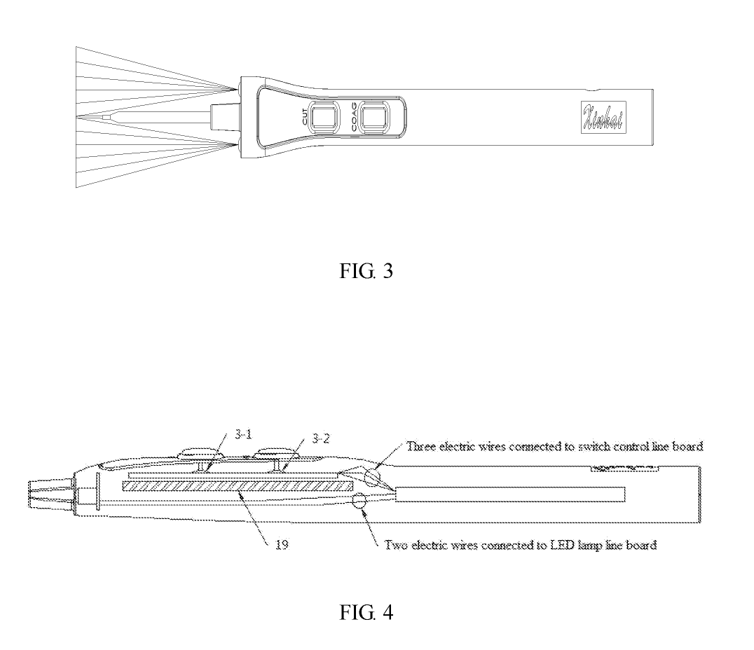

The specific working principle of a telescopic light source electrosurgical pencil as illustrated in FIG. 1 is as follows: High frequency and high voltage signals provided by an electric cutter machine are output through the head of an electrode for surgical operations, and form a loop with negative plates connected to a patient; in this way, high frequency currents in high current density are aggregated at the head of the electrode, and the tissue in contact with a point of the effective electrode tip is directly destroyed and operations of electric coagulation and electric cutting are performed on the affected parts. The specific structure of the electrosurgical pencil comprises: an electrosurgical pencil handle 4, wherein one end of the electrosurgical pencil handle 4 is connected to a cutter head 1, and two sides of a joint by which the electrosurgical pencil handle 4 is connected to the cutter head 1 are both provided with an LED lamp I 2-1 and an LED lamp II 2-2, the LED lamp I 2-1 and the LED lamp II 2-2 being parallelly arranged on the electrosurgical pencil handle 4. The parallelly arranged luminous sources make the luminance of the light emitting sources brighter. Therefore, two parallelly arranged LED lamps make a cutter head tip illuminate, which is convenient for the usage of a user. An inventive concept of the product lies in that two luminescent lamps are parallelly arranged on two sides of the cutter head 1, which are capable of generating 3000-500 Ixes power beam, and providing a clearer surgical view. The life cycle of a luminescent lamp is 10 thousand hours, which meets endurance requirement of the surgical operations.

The electrosurgical pencil handle 4 is provided with an electric cutting switch 3-1 and an electric coagulation switch 3-2, the other end of the electrosurgical pencil handle 4 is provided with an LED lamp switch 5 for controlling working of the LED lamps 2; and the electrosurgical pencil handle 4 is further connected to a cable 6, wherein the cable 6 is connected to a plug 7 connected to an electric cutter machine and a power adapter connector 9. The plug is directly connected to the power supply, AC is used in the power supply, and a stop switch is arranged, which may implement clear illumination at any time.

Further, the electrosurgical pencil handle 4 is internally provided with a power line 8 which collaboratively works with the LED lamp I 2-1 and the LED lamp II 2-2, wherein the power line 8 is connected to the power adapter connector 9 via the cable 6. In the working status, the plug 7 may be connected to the electric cutter machine by using a three-hole socket, the power adapter connector 9 is connected to a power adapter, the electric cutter machine provides electric coagulation and electricity cut signals, and the power adapter provides power supply for the LED lamps. At present, the requirement of the surgical operation circles on the definition of the surgical vision during the surgical operation is higher and higher, such that the requirement of precise surgical operation is implemented, the surgical quality is better and better, postoperative complications are reduced, and the requirement of life quality of people is satisfied. The solution at the market is that the luminance of the shadowless lamps is enhanced, and an LED lamp is used to replace the initial white light lamp. The market price of a set of shadowless lamps is between two hundred thousand and five hundred thousand Chinese Yuan. However, for some complicated surgical operations, such as thoracic surgical operation, neurosurgery surgical operation, the problem of clear surgical vision is still not solved. The parallelly set LED lamps disclosed in the present invention give great help for the doctors.

Further, the power line 8 is capable of integrating a DC power supply line and high frequency electric coagulation and electric cutting loops by using a five-core electric wire structure, and the power line 8 is lead out as a plug 7 having a three-core electric wire structure and a power adapter connector 9 having a two-core electric wire structure. A shunt portion may be set at a joint between the plug 7 and the power adapter connector 9.

Further, the electrosurgical pencil handle 4 is internally configured with a PCB board, and the electric cutting switch 3-1 and the electric coagulation switch 3-2 are capable of controlling working of the electric coagulation and electric cutting by controlling connection or disconnection of wiring of the PCB board. In the working status, the electric cutting switch 3-1 is pressed when the cutting is desired, such that the electric cutting switch 3-1 is conducted with the PCB board; in this way, the electric cutting function is implemented, and the end of the electric cutting is implemented when the electric cutting switch 3-1 is released. The electric coagulation switch 3-2 is pressed when the blood coagulation is desired during the point cutting, such that the electric coagulation switch 3-2 is conducted with the PCB board; in this way, the electric blood coagulation function is implemented. After the electric blood coagulation is completed, the electric coagulation switch 3-2 is released.

Further, as illustrated in Fl. 3 and FIG. 4, to make internal circuit structure of the electrosurgical pencil handle 4 simple and prevent the problems of complicated circuits, wound wire routing and unclear wiring, an isolation plate 19 is arranged inside the electrosurgical pencil handle. To be specific, the isolation plate 19 for isolating the electric coagulation and electric cutting lines from the LED lamp DC loop is arranged at the electric cutting switch 3-1 and the electric coagulation switch 3-2 on the electrosurgical pencil handle 4.

Further, the cutter head is capable of telescoping within a range of 46 mm to 130 mm, and the cutter head is capable of rotating within 360 degrees, which is convenient for the usage of the surgeon at any angles. The cutter head is capable of telescoping within a range of 46 mm to 130 mm, which is capable of adapting to various depth surgical operations, is convenient for the operation of the surgeon, and may reduce trouble of frequently changing cutter heads when the surgeon uses the cutter heads.

Further, the cutter head 1 is capable of rotating using a longitudinal axis of the electrosurgical pencil handle 4 as a rotation axis. The cutter head 1 is capable of cutting in the form of being perpendicular to a surface of a cut body, and forming at least one cut on the surface. The cutter head 1 is capable of rotating at the at least one cut, and maintaining perpendicular to the surface. During the surgical operation, the surgeon needs to manually press the electric cutting switch 3-1 and the electric coagulation switch 3-2. The cutting from top to bottom is the most convenient way according to gesture of holding a handle. If the transverse cutting, for example, from left to right, needs to be performed, the direction of the cutter head may be manually rotated at this moment with no need of rotating a wrist to complete the transverse cutting or the cutting at any other angles, which is the importance why the cutter head is perpendicular to a surgical surface.

Further, as illustrated in FIG. 2, a front end of the cutter head 1 is provided with a non-stick coating, which greatly reduces scab phenomenon of similar products in the process of usage, decreases times of cleaning cutter heads of surgeons, and cuts down surgical time. The cutter head 1 is provided with an insulating sleeve 10, which provides protection to the cutter head 1. A heat insulating Teflon sleeve 11 is arranged between the insulating sleeve 10 and the cutter head 1.

The telescopic light source electrosurgical pencil disclosed in the present invention is in a new design and in a simple structure, and uses parallelly set LED lamp I 2-1 and the LED lamp II 2-2 to corporately work, which provides stable enough luminance, and brings convenience to the surgical operation.

The above embodiments are used only for illustrating the present invention, but are not intended to limit the protection scope of the present invention. Various modifications and replacements readily derived by those skilled in the art within technical solution and inventive concept of the present invention shall fall within the protection scope of the present invention.

What is claimed is:

1. A telescopic light source electrosurgical pencil, comprising an electrosurgical pencil handle in which a control circuit is internally configured, one end of the electrosurgical pencil handle being connected to a cutter head, two sides of a joint by which the electrosurgical pencil handle is connected to the cutter head being both provided with an LED lamp I and an LED lamp II, the LED lamp I and the LED lamp II being parallelly arranged on the electrosurgical pencil handle, the electrosurgical pencil handle being provided with an electric cutting switch and an electric coagulation switch, the other end of the electrosurgical pencil handle being provided with an LED lamp switch for controlling working of the LED lamps, the electrosurgical pencil handle being further connected to a cable, the cable being connected to a plug connected to an electric cutter machine and a power adapter connector.

2. The telescopic light source electrosurgical pencil according to claim 1, wherein the electrosurgical pencil handle is internally provided with a power line which collaboratively works with the LED lamp I) and the LED lamp II, the power line being connected to the power adapter connector via the cable.

3. The telescopic light source electrosurgical pencil according to claim 2, wherein the power line is capable of integrating a DC power supply line and high frequency electric coagulation and electric cutting loops by using a five-core electric wire structure, and the power line is lead out as a plug having a three-core electric wire structure and a power adapter connector having a two-core electric wire structure.

4. The telescopic light source electrosurgical pencil according to claim 2, wherein the electrosurgical pencil handle is internally configured with a PCB board, and the electric cutting switch and the electric coagulation switch are capable of controlling working of the electric coagulation and electric cutting by controlling connection or disconnection of wiring of the PCB board.

5. The telescopic light source electrosurgical pencil according to claim 4, wherein an isolation plate for isolating the electric coagulation and electric cutting lines from the LED lamp DC loop is arranged at the electric cutting switch and the electric coagulation switch on the electrosurgical pencil handle.

6. The telescopic light source electrosurgical pencil according to claim 1, wherein the cutter head is capable of telescoping within a range of 46 mm to 130 mm, and the cutter head is capable of rotating within 360 degrees.

7. The telescopic light source electrosurgical pencil according to claim 6, wherein the cutter head is capable of rotating using a longitudinal axis of the electrosurgical pencil handle as a rotation axis.

8. The telescopic light source electrosurgical pencil according to claim 6, wherein the cutter head is capable of cutting in the form of being perpendicular to a surface of a cut body, and forming at least one cut on the surface.

9. The telescopic light source electrosurgical pencil according to claim 8, wherein the cutter head is capable of rotating at the at least one cut, and maintaining perpendicular to the surface.

10. The telescopic light source electrosurgical pencil according to claim 1, wherein a front end of the cutter head is provided with a non-stick coating, the cutter head is provided with an insulating sleeve, and a heat insulating Teflon sleeve is arranged between the insulating sleeve and the cutter head.
